(19) United States
(12) Patent Application Publication
(10) Pub. No.: US 2024/0382776 A1
(43) Pub. Date: Nov. 21, 2024

(54) Jewelry With Embedded Rotatable Magnets

(71) Applicant: Jeremy Madvin, Westlake Village, CA (US)

(72) Inventor: Jeremy Madvin, Westlake Village, CA (US)

(21) Appl. No.: 18/198,856

(22) Filed: May 18, 2023

(51) Int. Cl. A61N 2/06

(52) U.S. Cl. CPC A61N 2/06

(57) ABSTRACT

This disclosure relates generally to a novel form of jewelry with embedded, a manually rotatable magnet or multiple magnets that spin to throw the magnetic field some additional distance from the jewelry for pain relief, wellness, or other purposes. The magnets can have any polar alignment (negative side up, positive side up, alternating poles, or any other configuration). The jewelry can be placed at strategic points on the body for therapeutic purposes, wherein once the jewelry and attached magnet or magnets spin, the further traveling magnetic waves pass more deeply through the adjacent body and provide better wellness effects.

### COPYRIGHT NOTICE

A portion of the disclosure of this patent document contains material which is subject to copyright protection. The copyright owner has no objection to the facsimile reproduction by anyone of the patent disclosure, as it appears in the Patent and Trademark Office patent files or records, but otherwise reserves all copyright rights whatsoever.

### BACKGROUND

### Field of the Invention

The invention generally relates to the field of magnetic therapy. More particularly, the present invention relates to Jewelry with Manually Rotatable Embedded Magnets.

### Description of the Related Art

It is believed that magnetic energy works under the same principles as the ancient Chinese practice of acupuncture. Acupuncture, a traditional Chinese therapy, originated over three thousand years ago and is based on the belief that health and the body's overall well-being are determined by a balanced flow of “Chi”, the vital life energy present in all living organisms. According to the acupuncture theory, “Chi” circulates in the body along twelve major energy pathways called meridians, which are each linked to specific organs and body electrical systems. There is a specific relationship between acupuncture points, meridians, and the electrical currents of the body. The electricity is generated by an interaction of positively and negatively charged ions. As long as the flow of energy remains unimpeded and in balance, it is believed that we remain physically and mentally balanced, and therefore in good health.

Proper use of magnets for pain and other sources of unwellness has been proven to enhance the body's natural ability to heal itself, not just mask symptoms as many other treatments do. By using a properly designed and placed magnet for pain or illness, you can immediately start the healing process. It has been reported that biomagnets can be placed at strategic points on the body to positively influence pain control by directing magnetic fields to acupuncture points, acupuncture meridians, and the surrounding hard and soft tissue. For example, clothing that has discrete magnets attached thereto for producing healing effects at acupuncture points has been disclosed in prior art U.S. Pat. No. 5,720,046, which issued Feb. 24, 1998 to Lopez et al. Lopez discloses clothing and other personal wear which utilize discrete magnets to direct magnetic fields towards acupuncture points along the human torso, acupuncture points on the head along the temporal region, soft spot and uppermost vertebrae region at the base of the skull, and acupuncture points on the body appendages.

Prior art U.S. Pat. No. 6,427,486, issued Aug. 6, 2002 to Yellen, illustrates a pinless articulated band that is formed of a lineal array of metallic elements arranged in a repeating pattern. The elements are separably bonded to one another through magnetic forces acting therebetween. The band is characterized in that it is free of pins and other mechanical structural agents for interconnecting the band elements to one another. The configuration of the elements themselves is such that each has at least two surfaces, and such that these surfaces include one that is curved or arcuate. In a preferred embodiment of the invention, the elements include cylinders and discs that are of the same diameters. The cylinders and discs are so arranged that their axes are parallel to one another. In a closed loop configuration, the band exhibits aesthetic design features and finds use as a bracelet. The Yellen magnets are less powerful than those of the present invention and seem to require overlapping in order to stay together. There does not appear to be a healing intention for the Yellen patent.

Prior art U.S. Pat. No. 6,544,164, issued Apr. 8, 2003 to Fan, indicates a light wave energy health magnetic ring, which includes a plurality of blocks of different shapes formed from superconductive magnetic materials, natural crystals containing strong natural magnetic fields, and infrared ceramic beads. Bottom portions of the superconductive magnetic materials are formed with recesses for embedding crude crystals therein. Round, flat and circular-shaped, or wheel-shaped natural crystals and ceramic beads are spacedly disposed between adjacent superconductive magnetic materials such that the natural crystals are arranged in diagonally opposite positions. These elements are connected in a ring shape for wearing by a user. Magnetic waves can penetrate the muscles and bones of the body of the user to magnetize the cells of the body to thereby improve the circulation functions of the body.

Prior art U.S. Pat. No. 658,027, issued Sep. 18, 1900 to Steiger, indicates a therapeutic device that comprises magnetic disks provided with slots, several of which may be threaded on a tape for use in treatment. Alternatively, the disks may be used individually.

Prior art U.S. Pat. No. 5,806,346, issued Sep. 15, 1998 to Schlinger, provides a magnetic pendant necklace set method of manufacture. The method of inexpensively making a magnetic pendant necklace set relies upon affixing opposite-polarity magnets of at least 27 megagauss oersteds in pole strength to each of two, substantially-flat ornamental settings with protruding rings to which the opposite ends of any type of necklace can be attached. The ornamental settings function as a pendant clasp when joined together by their magnet centers and also as a secure mount for the easy attachment of magnet-studded, pendant ornaments.

Prior art U.S. Pat. No. 5,195,335, issued Mar. 23, 1993 to Hart, concerns a necklace with a flexible string that passes through a plurality of bead-like members, some of the beads on the string being magnetized so that when two of such beads are placed in the juxtaposed position, the necklace assumes a unique shape.

Prior art U.S. Pat. No. 4,912,944, issued Apr. 3, 1990 to Crosley, provides an article of jewelry that comprises a base element, which supports an ornamented substrate element thereon by magnetic attraction. Both the base element and the substrate element have magnetically attractable properties and at least one of them has the properties of a permanent magnet, whereby the substrate element is interchangeable with other like elements bearing different ornamentation.

Prior art U.S. Pat. No. 6,594,871, issued Jul. 22, 2003 to Hoffman, illustrates a jewelry element that includes a magnetic arrangement for ready substitution of multiple decorative elements. The element, which may be incorporated into a number of common jewelry pieces including a cuff link, earring, a stud for a tuxedo shirt, a ring or a pendant, includes a frame having a recessed central portion for receiving a decorative element. A pair of magnets is press-fit within a circular slot at the bottom of the recess and another magnet is adhesively fixed to the bottom of the decorative element. An aperture in the bottom of the recessed portion of the frame provides access for a stylus for selectively dislodging the decorative element.

Prior art U.S. Pat. No. 6,591,462, issued Jul. 15, 2003 to Fuhrman, is for a magnetic jewelry clasp that has two bodies and a safety catch. Each body has a surface magnetically attracted to the other. The clasp has a safety catch that includes an arm hinged to one of the bodies. The arm may be swung about the hinge between an open position and a closed position. A further magnetic attraction keeps the arm in the closed position until opened with a fingernail. A retaining wall may extend generally perpendicular to a direction of elongation of the arm to block a path that the other of the bodies could travel if the bodies were to separate from each other while the safety catch is in the closed position. If desired, a male connector may be inserted into a female connector to prevent separation of the bodies from each other. The male connector and female connector are usable in tandem with the retaining wall or in lieu thereof.

Prior art U.S. Pat. No. 5,732,451, issued Mar. 31, 1998 to Mars, concerns a device for magnetically attaching jewelry, name tags or the like to garments. The device includes inner and outer magnets and an anchor attached to the outer magnet. A pin-secured jewelry article or name tag is secured to the garment by passing the pin through a passageway in the anchor and then bringing the inner and outer magnets into magnetically close proximity on opposite sides of the garment.

What is needed is powerful magnetic properties that provide not only aesthetic value but deep-inside therapeutic effects as well. The current invention aims to provide jewelry articles wherein the magnet or magnets are built-in or attached to the jewelry item. Once the magnetic components housing the magnet or magnets or the magnets themselves spin, the magnetic field passes the human skin, deeper than a stationary, static magnet, and initiates a deep healing process.

It is to be noted that as per Applicant's knowledge, none of these prior art methods have been found to be completely suitable to meet these needs and are cumbersome and non-durable. Hence, the inventor of the present invention proposes to resolve and surmount existent technical difficulties and to eliminate the aforementioned shortcomings of prior art. Therefore, the object of the invention is an easy to use, efficient, fast, economical, durable, and safe mechanism.

### SUMMARY

In light of the disadvantages of the prior art, the following summary is provided to facilitate an understanding of some of the innovative features unique to the present invention and is not intended to be a full description. A full appreciation of the various aspects of the invention can be gained by taking the entire specification, claims, drawings, and abstract as a whole.

It is the objective of the invention to provide static magnets in a variety of shapes, sizes, and materials that are either built-in or are attached to the jewelry, wherein a variety of different types of jewelry, including bracelets, necklaces, charms, pendants, anklets, and rings can be used, to provide therapeutic as well as aesthetic value.

It is further the objective of the invention to provide components that are formed in a variety of shapes, including cubes, cylinders/rods, spheres, blocks, and squares to greatly vary the ornamental choices for assembling the magnets into jewelry of similar and mixed shapes.

It is furthermore the objective of the invention to provide magnetic jewelry wherein once the jewelry, or mechanism within the jewelry spins, the inbuilt magnet or magnets spin and release the longer, distance-traveling magnetic field that provides a deeper healing effect.

Therefore, it can be seen that the right combination of features incorporated in the device of the present invention produces surprising results that are not produced by the prior art.

This Summary is provided merely for the purposes of summarizing some example embodiments, so as to provide a basic understanding of some aspects of the subject matter described herein. Accordingly, it will be appreciated that the above-described features are merely examples and should not be construed to narrow the scope or spirit of the subject matter described herein in any way. Other features, aspects, and advantages of the subject matter described herein will become apparent from the following Detailed Description, Figures, and Claims.

### BRIEF DESCRIPTION OF THE DRAWINGS

The accompanying figures, where like reference numerals refer to identical or functionally similar elements throughout the separate views, together with the detailed description below, are incorporated in and form part of the specification, and serve to further illustrate embodiments of concepts that include the claimed invention and explain various principles and advantages of those embodiments.

FIG. 1 shows the front view of the present invention.

FIG. 2 shows a close-up view of the present invention.

FIG. 3 shows another perspective view of the complete assembly.

FIG. 4 is another close-up view of the present invention.

FIG. 5 is another view of the present invention.

The apparatus and method components have been represented where appropriate by conventional symbols in the drawings, showing only those specific details that are pertinent to understanding the embodiments of the present invention so as not to obscure the disclosure with details that will be readily apparent to those of ordinary skill in the art having the benefit of the description herein.

### DETAILED DESCRIPTION

Detailed descriptions of the preferred embodiment are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in virtually any appropriately detailed system, structure, or manner.

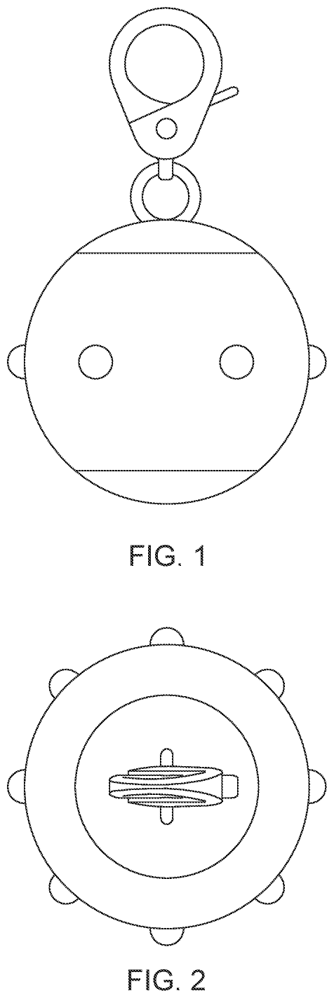

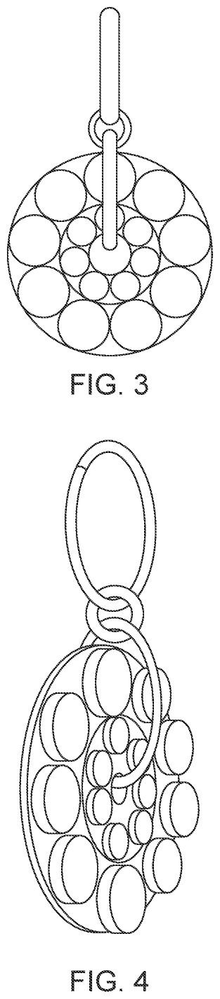

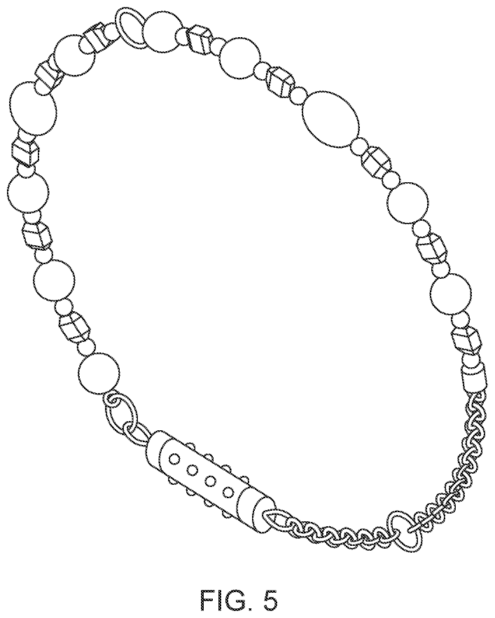

FIGS. 1-5, discloses jewelry would have a magnet or magnets built into it or onto it. The magnets can be either neodymium, ferrite, or any other kind of magnet. The optional finish may comprise finishes including silver, nickel, black epoxy, 24k electro-plated gold, platinum, or black nickel, which may be intermixed in a piece of magnetic jewelry for a wide variety of finish looks that can be worn together in different combinations.

Each of the magnets is diametrically magnetized utilizing a north/south relationship. Each of the components may have a shape consisting of a cube, a cylinder or rod, a sphere, a rectangular block, or some other shape. The shapes can be used together in a single piece of magnetic jewelry or as a component within a spinning mechanism of the jewelry for a variety of decorative choices.

The piece of magnetic jewelry creates a therapeutic magnetic field around a portion of a user wearing the piece of magnetic jewelry. The piece of magnetic jewelry may be assembled into a configuration and worn as a bracelet, a necklace, an anklet, a ring, or another form of jewelry. It is furthermore the objective of the invention to provide magnetic jewelry wherein once the jewelry or component housing the magnet or magnets spin, the inbuilt magnet spins and releases a longer, distance-traveling magnetic field that provides a deeper effect than non-rotating static magnets.

The magnetic piece of jewelry may be worn to create a therapeutic magnetic field around a portion of a user wearing the jewelry and possibly enhance the body's natural ability to heal itself. The jewelry assembled must include a magnet or magnets measurable in Gauss and can be placed at strategic points on the body to positively influence pain and other forms of unwellness by directing magnetic fields to acupuncture points, acupuncture meridians, and the surrounding hard and soft tissue.

The working of the current invention can be discussed by understanding the example of a pendant wherein the jewelry item is not only designed as a pendant but as a fidget widget. An example of such an assembly includes four magnets with alternating poles and can be designed in a variety of shapes. Once the assembly is spined, it throws the magnetic field and the magnetic field will travel farther and pass through a deeper portion of an adjacent body than a stationary, static magnet.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separately claimed subject matter.

1. Magnetic jewelry, including a magnet or magnets for therapeutic and decorative purposes, the system comprising:
A magnet or series of magnets, each having a magnetic force, and each of the magnets can be neodymium, ferrite, or any other kind of magnet, optionally having a coating of and having a form and an exterior finish appropriate for use in magnetic jewelry;
Wherein, once the magnetic jewelry spins, the inbuilt magnet or magnets spin and release the longer, distance-traveling magnetic field that provides a deeper healing effect than non-rotating static magnets.
Wherein the piece of magnetic jewelry creates a strong therapeutic magnetic field around and passes deeper into or through the user wearing the piece of magnetic jewelry.

2. The system of claim 1 wherein each of the magnets has any shape selected from the list of shapes consisting of, but not limited to, a cube, a cylinder, a rod, a sphere, and a rectangular block, and the shapes can be used together in a single piece of magnetic jewelry for a variety of decorative choices.

3. The system of claim 1 where the piece of magnetic jewelry is adapted for use as one of a list of pieces of jewelry consisting of a bracelet, a necklace, a pendant, a charm, an anklet, and a ring.
